(19) United States
(12) Patent Application Publication
(10) Pub. No.: US 2023/0172196 A1
(43) Pub. Date: Jun. 8, 2023

(54) A BIODEGRADABLE NANO - ENCAPSULATED DISINFECTANT COMPOSITION

(71) Applicant: NANOKSIA BIYOTEKNOLOJI SANAJI TICARET ANONIM SIRKETI, Istanbul (TR)

(72) Inventor: Kursat Seyfi DEMIREZEN, Kayseri (TR)

(21) Appl. No.: 17/618,941

(22) PCT Filed: Dec. 29, 2020
(86) PCT No.: PCT/TR2020/051432

§ 371 Date: Dec. 14, 2021

(51) Int. Cl. A01N 25/28; A01N 59/16; A01P 1/00

(52) U.S. Cl. CPC A01N 25/28; A01N 59/16; A01P 1/00; B82Y 5/00

(57) ABSTRACT

A disinfectant composition contains myrrh tree plant extract, highbush cranberry leaf extract, sumac leaf extract, juniper fruit extract, jasmine and/or daisy flower extract, apple and sunflower face pomace, silver nanoparticle and pure water. In particular, disclosed is a biodegradable nano-encapsulated vegan and ecological disinfectant composition containing herbal synthesized silver nanoparticles which is effective in providing personal hygiene with general and public areas, especially the health sector being in the first place, and a production method thereof.

### TECHNICAL FIELD

The invention relates to a herbal synthesized, biodegradable nano-encapsulated disinfectant composition comprising silver nanoparticles.

The invention relates in particular to a vegan and ecological disinfectant composition having antibacterial, antiviral and antifungal effects, and comprising herbal synthesized silver nanoparticles, and production method thereof, in providing personal hygiene in the general and public areas with the health sector being in the first place.

### PRIOR ART

Currently used hygienic formulations comprise totally synthetic origined, toxic, chemical substances harmful to human and environmental health. This kind of synthetic chemicals adversely affect human and environmental health. In the existing formulations, quaternary ammonium compounds, chlorhexidine, iodine compounds or alcohol based iodine, iodophores, para-chloro-metha-xylenol (PCMX), hexachlorophene and octenidin dihydrochloride, chlorine derivatives, etc. are used in providing antimicrobial efficacy.

As a result of this, the developed products cause respiratory toxicity and sensitivity on the skin, allergy and irritation. The fact that skin protective, softener and humidifiers added to the formulations in order to overcome this problem are synthetic chemicals, threatens human health in an increasing manner.

In prior art, in the compositions which are being used as nanotechnological disinfectants, silver (Ag) nanoparticles obtained by chemical or physical synthesis are being used as active matter. Chemical reduction by using organic solvent or water is the most common technique in the production of silver nanoparticles (AgNPs). As reducing agent, generally boron hydride (borohydride), sodium dodecyl sulfate, sodium citrate, ascorbate polyols, NaBH4, N2H4 and N, N-dimethylformamide are used. In the end, colloidal silver subjected to chemical processing and diameter thereof reduced to nano level is being obtained. A great majority of chemical reducing reactions require high heat shock application. For obtaining chemical Ag+in thermal way, m-hydroxyl benzaldehyde (m-hydroxy benzaldehyde), hydrazine and dextrose are used together as the reducing agent. Therefore, morphology of the nanoparticles is effected by the value of temperature applied during the synthesis. Furthermore, sodium formaldehyde sulfoxylate is being used too as the secondary reducing agent. And in another method, glucose and gluconic acid are being used. Among the alternative methods, method of subjecting to X-ray radiation in which ethylene glycol—water mixture is used can be mentioned. In this method, nanoparticles are obtained by using UV rays. Especially for the synthesis of factory—made silver nanoparticles, pyrolysis technique too implemented at high temperature and pressure in furnaces set at 720° C. is another known method. For this method Ag/MgO mixture is needed, and in the final stage ammonium chloride (NH4Cl) solution is used to remove the MgO.

For example, in application number EP2309863B1, the concentrate to prepare the disinfectant and methods for manufacturing thereof are disclosed. The invention relates to an aqeuous concentrate which will be stable during storage, and is for preparing a disinfectant. The concentrate comprises the following: hydrogen peroxide at a concentration between 30% and 70% by volume of the final concentrate; colloidal silver at a concentration between 150 and 1000 ppm by weight of the final concentration; a stabiliser selected from the group consisting of gum arabic, gelatine, guar gum, carrageenan, pectin and combinations thereof, the concentration of the stabiliser is between 10 and 100 ppm by weight of the final concentrate; and phosphoric acid to adjust the pH value of the concentrate at 3 or lower.

And in application number EP3184492B1, the invention falls among the antiseptic formulations including the disinfectants targeted to disinfection of water for health and hygiene; and for disinfection of water especially in swimming pools and in other artificial reservoirs; for health and hygienic purification of rooms, household equipment, furniture, home appliances and industrial devices and also rinse and waste water. An antiseptic formulation encompasses both silver and also particles of very small size encompassing silver chloride. An antiseptic formulation can encompass additionally at least one amphoteric surfactant. In order for disinfection of water to be implemented, very small sized particles encompassing silver and silver chloride are added into the water at least once.

And in national patent application number TR2016/20278, a solution comprising colloidal silver is disclosed. This invention relates in general to a kind of water which has been developed for the purpose of elimination of ailments such as healing of wounds, skin beauty (anti aging), disinfectant, elimination of pathogens in the body and comprising colloidal silver which offers advantages while not harming useful bacteria with respect to synthetic antibiotics.

Consequently, in compositions produced by the known methods, there are silver nanoparticles obtained by one or a few of the techniques explained above. Also, in the existing methods, it is necessary to add the surfactant to the medium during chemical synthesis silver for the stabilisation of nanoparticles. The compositions of the existing methods have been supported with substances of synthetic or chemical origin which could be risky as far as human and environmental health are concerned. Furthermore, the cost of silver nanoparticles with chemical synthesis depending on the chemical required for the application, in other words energy requirement varies. Time spent in the processes is long.

Also, in the existing products comprising nanotechnological silver; yellow, brown, black staining is being observed on the applied surfaces.

Encapsulation is known as a process in which a substance is confined inside another substance. While the substance being coated is called as the core, fill or internal phase; the coating substance is generally called as the coating, membrane, shell, capsule, carrier material, external phase or matrix. In many cases, encapsulation means a technology with which bioactive ingredients are surrounded completely with a physical barrier, coated and protected.

One of the most important reasons of encapsulation process of the active substances in the scope of the invention is to provide improved stability during processing and in the final product. Active substance is generally inside a molecule or is in a nanosized state. The main benefit is the uniformity offered by the nanosize which this case causes a better encapsulation activity in the material as well as better physical and chemical properties.

The invention in application number US005503781A belongs to obtaining microcapsules formed from the copolymer comprising vinyltoluenesulfonic acid and maleic acid anhydrate. The existing method comprises toluene derivative which has toxic cancerogen effect in terms of human and environmental health. Its interaction with active substances having antimicrobial effect is indefinite. Also, its cost and thus energy consumption is much depending on the chemical in microencapsulation with chitosane being in the first place.

The invention in application number WO 2019/046978 A1 belongs to obtaining microcapsule, specifically directed to obtaining carrier system for carotenoids. Here, the subject is the treatment of lecithin which is an animal protein with ethyl alcohol in obtaining it as the capsule (core). In this case, it prevents the product to be vegan.

The invention in application number WO2002058812 belongs to obtaining hepafilter with n-alkyldimethyl benzylammonium saccharinate biocide active substance. Microcapsule dimension is in the size of 50-500 micrometers, the effect of the active substance is being reduced.

It has been necessary to perform an improvement in the related technical field due to the negativities narrated above and because of the inadequacy of existing solutions on the subject, and due to the fact that it uses dangerous chemicals in terms of environmental and human health for obtaining it.

### BRIEF DESCRIPTION OF THE INVENTION

The present invention relates to a biodegradable nano-encapsulated disinfectant composition of which the active substance is herbal synthesized silver nanoparticles and its method of production which meet the above mentioned requirements, which eliminate all disadvantages and which offer some additional advantages.

The prioritized object of the invention is to provide solution to eliminate the problems which will adversely affect the human and environmental health in use according to the formulations with chemical matter content that are used in the existing technique with the nanocapsule comprising the active substance obtained by vegan synthesis in the composition of the invention. In order to provide this solution, the mixture consisting of sunflower face pomace, highbush cranberry, myrrh, sumac, jasmine or daisy and juniper plants has been used in the invention.

Another object of invention is to set forth that chemical substances are not required to be used as reducers and stabilizers as in the prior art thanks to the sufficiency of the plant extract as reducer in obtaining silver nanoparticles.

Another object of invention is to set forth that hydrogen peroxide, quaternary ammonium compounds, and other harmful chemicals are not required to be used as supporting active substance as in the prior art thanks to vegan synthesis silver nanoparticles, and thanks to the sufficiency of the sole active substance having microbial effect.

Another object of invention is to reduce the time, production cost and energy requirements and to facilitate production stages in removal of pathogen microbes from the environment with the biological synthesis composition of the invention. The composition obtained by using vegan extract has antimicrobial efficacy in gram positive and gram negative bacteria comprising silver nanoparticle, and in fungi, viruses, spores, mycobacteria and bacteriophages.

Another object of invention is to enable the composition to give off distinctive pleasant scent by using jasmine/daisy saps, apart from its contribution to the synthesis of silver nanoparticles. It is also to set forth the usability of jasmine or daisy saps in lieu of substances such as glycerine and vitamin E used in prior art to impart humidification in leather.

Another object of invention is to set forth an ecological disinfectant experimentally proven to be not causing toxic effects in the studied aquatic living creatures in the ecotoxicity studies carried out with silver nanoparticles synthezised from plant extract.

Another object of invention is to set forth a vegan disinfectant composition which does not harm the respiratory system and which does not cause allergy on the skin thanks to silver nanoparticles being synthesized with vegetative method. It can be applied to all kinds of environments in which the human being is in touch with, and it is environmentally friendly.

Another object of invention is to set forth an effective disinfection composition which is not affected from environmental changes, which is physically more stable, which maintains its odor for a long time, which has increased permanence effect on the surface which it has been applied thanks to apple and sunflower face pomace, and thanks to jasmine/daisy saps and silver nanoparticles surrounded by nanocapsule obtained from biodegradable pectin.

Another object of invention is to set forth the usability of organic lactic and citric acid which are EPA approved, harmless to environment and humans in obtaining biodegradable nanocapsules instead of harmful solvents such as sodium citrate, ethanol, hydrochloric acid, oxalic acid, nitric acid.

Another object of invention is to set forth a disinfectant which can be applied to the surface in different ways such as spraying, immersion, direct application to surface and which does not leave stains on the surface thanks to technique of reducing the used pure silver to nanosize. The fact that nitrate form of silver is being used in nanosilver compositions of prior art causes the final product to leave stains on the surface which it has been applied.

In order to achieve the objects mentioned above, the invention is a nano-encapsulated disinfectant composition comprising herbal synthesized silver nanoparticles, and it comprises myrrh tree plant extract, highbush cranberry leaf extract, sumac leaf extract, juniper fruit extract, jasmine and/or daisy flower extract, apple and sunflower face pomace, silver nanoparticle and pure water.

According to one embodiment of the invention, it comprises 1-2.5% by weight myrrh tree plant extract, 0.7-1.3% by weight highbush cranberry leaf extract, 0.6-2.2% by weight sumac leaf extract, 0.3-0.9% by weight jasmine and/or daisy flower extract, 2-4.5% by weight juniper fruit extract, 0.74-1.2% by weight apple pomace, 0.9-1.4% by weight sunflower face pomace, 0.037-0.059% by weight silver nanoparticle and 86-93% by weight pure water.

According to one embodiment of the invention, it comprises 2.067% by weight myrrh tree plant extract, 1.1% by weight highbush cranberry leaf extract, 1.5% by weight sumac leaf extract, 1% by weight jasmine or daisy flower extract, 3.4% by weight juniper fruit extract, 0.98% by weight apple pomace, 1.25% by weight sunflower face pomace, 0.044% by weight silver nanoparticle and 88.659% by weight pure water.

In order to accomplish the above mentioned objects, the invention relates is a production method of nano-encapsulated disinfectant composition comprising herbal synthesized silver nanoparticles; and comprises the process steps of:
i. Brewing myrrh tree, highbush cranberry leaf and sumac leaf in water,
ii. Extraction of juniper fruits in water,
iii. Extraction of jasmine and/or daisy plants in water/vegetative alcohol,
iv. Gelling apple and sunflower face pomace in water, obtaining pectin with lactic and citric acid
v. Preparation of solution with pure silver, acetic acid and hydrogen peroxide,
vi. Mixing the prepared solution with the vegetative extracts obtained in process steps (i), (ii), (iii),
vii. Coating the nanoparticle obtained in step (vi) with the nano-capsule material obtained in step (iv).

According to one embodiment of the invention, process step (i) comprises brewing Myrrh tree plant in pure water first at 65-75° C. for 20-25 minutes by occasionally mixing; brewing at 65-85° C. for 10-15 minutes by occasionally mixing by adding equal amounts of powdered Highbush cranberry leaf and Sumac plants over it; subjecting to ultrasonification process in an ultrasonic device at 40° C. for 10-15 minutes for increasing the speed of extraction; shock cooling of the obtained extract and subjecting to ultrasonification process for 10-15 minutes; subjecting to shock cooling process and centrifuging at +1° C. at 4500-5500 rpm for 10-15 minutes and storing at +4° C.

According to one embodiment of the invention, process step (ii) comprises heating Juniper fruits in pure water first at 85-95° C. for 20-30 minutes; subjecting to ultrasonification process for 10-15 minutes, then leaving it to fermentation in the closed container at 45-55° C. for 5-10 days; shock cooling of the obtained extract; and centrifuging at +1° C. at 4500-5500 rpm for 10-15 minutes and storing at +4° C.

According to one embodiment of the invention, process step (iii) comprises the processes of brewing jasmine (and/or daisy) flowers first at 35-45° C. for 5-10 minutes by adding 54% vegetative alcohol; then at 50-60° C. for 20-30 minutes by adding pure water by occasionally mixing; cooling in ice bath by subjecting to shock cooling and subjecting the cooled extract to ultrasonification process for 10-15 minutes in ultrasonic device; centrifuging at +1° C. at 4500-5500 rpm for 5-7 minutes; separation of solid and liquid extract parts by filtering through filter paper having 11 μm pore diameter.

According to one embodiment of the invention, process step (iv) comprises the processes of putting 200-350 grams of apple pomace and adding 20-30 ml of pure water over it; smashing for 1-3 minutes, mixing at 250-300 rpm to bring to boiling point at 80-90° C. for 25-30 minutes; adding citric and lactic acid over it and mixing and cooling; centrifuging at 5300 rpm for 10-15 minutes and filtering through filter paper and leaving at 60-80° C. for 20-25 minutes.

According to one embodiment of the invention, process step (iv) comprises the processes of putting 150-250 grams of sunflower face pomace and adding 400-500 ml of pure water over it; heating at 90-95° C. for 35-40 minutes; addition of citric and lactic acid and mixing continuously at 250-350 rpm and smashing for 1-3 minutes; cooling for 20-30 minutes and centrifuging at 5300 rpm for 10-15 minutes; filtering through filter paper and heating at 30-40° C. for 20-25 minutes.

According to one embodiment of the invention, process step (v) comprises the processes of weighing pure silver in the ratio of 0.037-0.059% and treating with acetic acid and hydrogen peroxide in the ratio of 0.5:2; adding the extracts obtained in process steps (i) (ii) to the solution in bottle number 5 and mixing at 750-850 rpm for 5-10 minutes, and addition of the extract obtained in process step (iii) and mixing at 750-850 rpm for 20-30 minutes.

According to one embodiment of the invention, process step (vii) comprises the processes of taking the supernatant obtained in process step (iv) from apple pomace and adding dropwise slowly to the mixture obtained in process step (v) which is mixed continuously at 40° C.

According to one embodiment of the invention, process step (vii) comprises the processes of taking the supernatant obtained in process step (iv) from sunflower face pomace and adding dropwise slowly to the mixture obtained in process step (v) which is mixed continuously at 40° C.

Structural and characteristic features of the invention and all of its advantages will be understood more clearly by means of the detailed description given below and therefore it should be appreciated by considering this detailed description.

### BRIEF DESCRIPTION OF FIGURES

FIG. 1 is a graph showing the nanosilver size distribution in the composition of the invention.

FIG. 2 is a graph showing the Zeta potential in the composition of the invention.

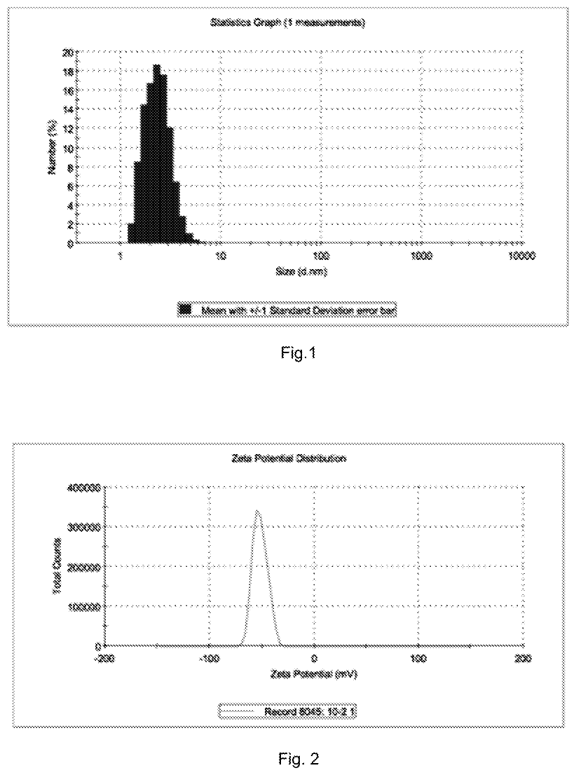

### DETAILED DESCRIPTION OF THE INVENTION

In this detailed description, disinfectant composition of the invention and production method thereof are described aimed at only better understanding of the subject and without forming any limiting effects.

The invention relates to a biodegradable nanocapsule disinfectant composition comprising herbal synthesized silver nanoparticle and the production method of this composition.

The disinfectant composition of the invention in its most fundamental form comprises myrrh tree extract, highbush cranberry leaf extract, sumac plant extract, juniper fruit extract, jasmine and/or daisy flower extract, apple and sunflower face pomace, silver nanoparticle and pure water.

TABLE 1
The content of the composition of the invention

| | Preferred | Usable |
|---|---|---|
| | amount by | amount by |
| Content | weight (%) | weight (%) |
| Myrrh (Commiphora myrrha) tree extract | 2.067 | 1-2.5 |
| Highbush cranberry (Viburnum opulus) leaf | 1.1 | 0.7-1.3 |
| extract | | |
| Sumac plant (Rhus coriaria) extract | 1.5 | 0.6-2.2 |
| Jasmine (Jasminum officinale L.) | 1 | 0.3-0.9 |
| flower extract and/or | | |
| Daisy (Anthemis nobilis) flower extract | | |
| Juniper (Juniperus communis) fruit extract | 3.4 | 2-4.5 |
| Apple (Malus domestica) pomace | 0.98 | 0.74-1.2 |
| Sunflower (Helianthus annus) face pomace | 1.25 | 0.9-1.4 |
| Pure silver | 0.044 | 0.037-0.059 |
| Pure water | 88.659 | 86-93 |

Highbush cranberry plant is known for its antioxidant, antispasmodic, antiinflammatory, antiallergic, sedative and diuretic effects and is being used in the treatment of many diseases kidney diseases being at the first place. Boiled highbush cranberry can be used in the treatment of many diseases such as asthma, rheumatism, high blood pressure, epileptic episodes (epilepsy), mumps, spasms after delivery, sleep disorder. Highbush cranberry extract is also good for gall bladder disease and some liver diseases. There are also studies related to positive effects of highbush cranberry in reducing some cancer tumors.

In the composition of the invention, secondary ingredients such as valeric acid and oxalates that highbush cranberry plant comprises develops synergy with the other plant active substances that are being used together with it and reduces the synthesized nanosilver particle size to around 5-25 nm.

Myrrh tree extract has been helpful in remaining stable of the nanoparticles in the formulation. Myrrh tree consists of etheric oil in the ratio of 10 percent, resin and sticky mass in the ratio of 40 percent. Its antibacterial and antifungal effects are known.

In the composition of the invention, secondary ingredients such as limonen and mirsenol that myrrh tree extract comprises develops synergy with the other plant active substances that are being used together with it and reduces the synthesized nanosilver particle size to around 5-25 nm.

Sumac is helpful in the treatment of dermal problems and in healing of wounds with its anti-inflammatory, antibacterial and antimicrobial properties. Chewed sumac leaves are good for skin rush, gingivitis.

In the composition of the invention, sumac plant supports the antibacterial efficacy of the composition thanks to tanen and mirisetin that it comprises, and aids in reducing silver to the desired nanoparticle size.

Antimicrobial and antifungal effects of Jasmine and/or daisy flower are known, and it is used in skin replenishing.

In the composition of the invention, pleasant scent feature has been imparted to the composition thanks to addition of jasmine and/or daisy hydrosole. At the same time, humidifying feature in dermal usage has been imparted to the composition.

The resin, branches, leaves and fruits of juniper are employed in cosmetics and medicine thanks to its acidic content. Its antibacterial, antimicrobial, anti-inflamatuvar, antioxidant effects are known.

In the composition of the invention, secondary ingredients such as glycose and yunen which the juniper fruit extract comprises contribute to shortening of the time in obtaining nanosilver.

In the composition of the invention, apple pomace has imparted organic and biodegradable nanocapsule content property thanks to high cellulose and pectin that it comprises.

In the composition of the invention, sunflower face has imparted organic and biodegradable nanocapsule content property thanks to high cellulose and pectin that it comprises.

Silver is commonly used in photography industry, electronics, coin manufacturing, making ornaments and jewelries, alloys, dentistry. It is also being used in artificial raining, in making mirror tains, computer relay contacts, in making cells. Pure silver is also used in obtaining acetic acid, paints and photography substances. Likewise, silver in powder form is being used in new ceramic type coating to make glass and wood electrically conductive. Silver is a very powerful antibacterial with very low toxic. Because of this reason, it is used extensively in medical and textile fields.

In the composition of the invention, silver has been preferred as the active substance that provides antimicrobial effect to the invention. Obtaining disinfectant composition by the use of a single active substance in low amount has been achieved. Pure water is preferably at a purity of 1 microSiemens and it is effective in the formulation as a solvent. Instead of a solvent such as alcohol, acetone, etc. which is harmful and costly in terms of environment and human health, ecological and skin friendly pure water has been used as the solvent. In this manner, a product which does not cause dermal irritation and respiratory toxicity has been obtained with the composition of the invention.

The production method of the composition of the invention in its most fundamental form comprises the following process steps:
i. Brewing myrrh tree, highbush cranberry leaf and sumac leaf in water,
ii. Extraction of juniper fruits in water,
iii. Extraction of jasmine and/or daisy plants in water/vegetative alcohol,
iv. Gelling apple and sunflower face pomace in water, obtaining pectin with lactic and citric acid
v. Preparation of solution with pure silver, acetic acid and hydrogen peroxide,
vi. Mixing the prepared solution with the vegetative extracts obtained in process steps (i), (ii), (iii),
vii. Coating the nanoparticle obtained in step (vi) with the nano-capsule material obtained in step (iv).

In the preparation of a preferred embodiment of the invention, 7 separate lightproof glass bottles having a lid are prepared. Myrrh tree plant put in lightproof bottle number 1 having a lid in the amount with which the ratios in Table 1 are satisfied is brewed in pure water first at 65-75° C. for 20-25 minutes by occasionally mixing and extract is obtained. Equal amounts of powdered highbush cranberry leaf and sumac leaf are added over the myrrh extract which has been brewed in bottle number 1 for 20-25 minutes, and it is brewed at 65-85° C. for 10-15 minutes by occasionally mixing and the obtained extract is subjected to ultrasonification process in ultrasonic device at 40° C. for 10-15 minutes for increasing the speed of extraction. At the end of the period of time, it is cooled by subjecting it to shock cooling process and is centrifuged at +1° C. at 4500-5500 rpm for 10-15 minutes. The supernatant is taken and stored at +4° C.

10-15 grams of juniper fruits are put in lightproof bottle number 2 having a lid, and 50-80 ml of distilled water are added over it and it is brought to boiling point first at 85-95° C. for 20-30 minutes, after the extract has been subjected to ultrasonification process in the ultrasonic device for 10-15 minutes the lid of the bottle is closed and left to fermentation at 45-55° C. for 5-10 days. At the end of the period of time, the extract in bottle number 2 is centrifuged at +1° C. at 4500-5500 rpm for 10-15 minutes. The supernatant is taken and stored at +4° C.

Jasmine and/or daisy flowers put in lightproof bottle number 3 having a lid are brewed first at 35-45° C. for 5-10 minutes by adding 54% vegetative alcohol (ethanol); then brewed at 50-60° C. for 20-30 minutes by continuously mixing by adding pure water. At the same time, removal of alcohol that it comprises is achieved. The extract belonging to the mixture in bottle number 3 is cooled by subjecting it to shock cooling process in the ice bath and the cooled extract is subjected to ultrasonification process in the ultrasonic device for 10-15 minutes. The mixture cooled in bottle number 3 is centrifuged at +1° C. at 4500-5500 rpm for 5-7 minutes, is filtered through filter paper having 11 μm pore diameter and the remaining solid and liquid parts are separated.

The filtrate obtained from bottles number 1 and 2 are added to bottle number 4 in the ratio of 1.5:6.5 by volume and is mixed.

Preferably pure granule silver is weighed in the ratio of 0.037-0.059% by volume in lightproof glass bottle number 5 having a septum, and is treated with acetic acid and hydrogen peroxide in the ratio of 0.5:2 (100 ml has been taken as the baseline). In order to provide reduction of silver solution in bottle number 5 to nanosilver, 6.25-8.067 ml of plant extract are added from bottle number 4 and mixed at 750-850 rpm for 5-10 minutes. 1-2 ml of extract are taken from bottle number 3 and added to bottle number 5 and mixed for 20-30 minutes at 750-850 rpm. Acetic acid, hydrogen peroxide and alcohol are not present in the final product; alcohol is being removed prior to the synthesis, acetic acid and hydrogen peroxide are being removed during the synthesis since they are biodegradable.

200-350 grams of Apple pomace are put in lightproof bottle number 6 having a lid, and 20-30 ml of distilled water are added over it and is smashed with a laboratory type blender for 1-3 minutes, is brought to boiling point over the hot plate by mixing continuously at 250-300 rpm at 80-90° C. for 25-30 minutes. 0.5-1.2 ml of citric acid, 0.7-1.5 ml of lactic acid are added over it and its lid is closed and mixing is continued. It is cooled at +2° C. for 20-30 minutes. It is centrifuged at 5300 rpm for 10-15 minutes and filtered through Whatman No. 1 filter paper. The filtrate is left at 60-80° C. for 20-25 minutes.

150-250 grams of sunflower face pomace are put in lightproof bottle number 7 having a lid and 400-500 ml of distilled water are added over it and it is brought to boiling point at 90-95° C. for 35-40 minutes, 0.5-1.8 ml of citric acid, 1.2-2 ml of lactic acid are added and it is mixed continuously for 10-15 minutes at 250-350 rpm. It is smashed with a laboratory type blender for 1-3 minutes, cooled at +2° C. for 20-30 minutes. It is centrifuged at 5300 rpm for 10-15 minutes, and filtered through filter paper. The filtrate is heated at 30-40° C. for 20-25 minutes.

The supernatant in bottle number 6 is taken in the ratio of 0.74-1.2% by volume and added dropwise slowly to the mixture in bottle number 5 which is being continuously mixed at 40° C.

The supernatant in bottle number 7 is taken in the ratio of 0.9-1.4% by volume and added dropwise slowly to the mixture in bottle number 5 which is being continuously mixed at 40° C.

The product having daisy or jasmine scent, nanosilver active substance disinfectant effect coated with the obtained nanocapsule is filled in. The product is preferably supplied in spray boxes.

In the method of obtaining the composition of the invention, since all of the reducing, stabilizing agents and polymers are composed of herbal raw materials, the solution of avoiding negative results which can effect human and environmental health according to formulations with synthetic content used in prior art is proposed. Also, with the odor agents coated with nanocapsule has been proposed to prevent odor efficacy in the existing scented products getting lost in a short period of time. By using nanocapsule in the composition of the invention, enabling the active substance to be durable against varying environmental factors, and being physically more stable is being proposed. Also, enabling the capsule to be biodegradable by using organic substances instead of harmful chemicals in obtaining nanocapsule is being proposed. In order to provide this solution, Highbush cranberry leaf, Myrrh tree, Sumac leaf, Juniper fruit, Jasmine and/or daisy flower, apple and sunflower face pomace have been used in the invention.

In the method of obtaining the composition of the invention, by the synergistic effect by using Myrrh, Highbush cranberry, Juniper and Sumac extract create;
reducing pure silver to 5-25 nm size has been provided;
maintaining the obtained composition stable for a long time without addition of chemicals has been provided;
dermal allergy has been avoided,
respirable state has been achieved,
production time has been shortened.

In the method of obtaining the composition of the invention, by using pure silver;
active matter with antimicrobial effect has been obtained;
yellow, brown, black staining problem on the applied surfaces has been eliminated;
liberation of harmful chlorine and nitrate compounds in the content of the composition has been hindered.

In the method of obtaining the composition of the invention, by using jasmine or daisy saps;
composition with a pleasant scent has been provided;
humidifying effect in dermal application has been imparted to the composition.

In the method of obtaining the composition of the invention, by using Apple and Sunflower face pomace the following have been provided;
scent giving plants preserving its efficacy for a long time with the active substance,
obtaining organic origined, biodegradable coating substance,
enhancing the efficacy of the composition on the surfaces it is used;
composition remaining physically more stable.

Technical benefits which the disinfectant of the invention obtained as above provides superior to silver active disinfectants in the prior art are given below in Table 2.

TABLE 2
The properties of the invented disinfectant

| | The composition of | | Silver active disinfectants |
|---|---|---|---|
| | the invention | | in prior art |
| Silver source | At 999.9 purity | | AgNO₃ |
| Nanoparticle size | 5-25 | nm | 40-90 nm |
| Dermal irritation | None | | Present |
| Stabilizer | Plant | | Chemical |
| Protectiveness on the | 3-6 | months | A few hours |
| surface it has been | | | |
| applied | | | |
| Active substance | Silver | | Silver + hydogen |
| | | | peroxide |
| | | | Silver + quaternary |
| | | | ammonium compounds |
| Production time | 20-35 minutes after | | 2-8 hours according to |
| | the preparation stage | | the number of chemicals |
| | | | it has |
| Production cost | 60% less | | |
| Toxicity | None | | Present |
| Shelf life | 2 | years | 3 months-2 years |

With the active content of the composition of the invention, antibacterial, antiviral and anti fungal effects are provided. Results of the efficacy test of the composition of the invention are given in Table 3.

TABLE 3
Efficacies of bacteria, mycobacteria, fungi and viruses

| | Efficacy | Log removal | Action time | |
|---|---|---|---|---|
| Bacteria | | | | |
| E. coli | 99.999% | 5 Log | 1 | minute |
| S. aureus | 99.999% | 5 Log | 1 | minute |
| K. pneumonia | 99.999% | 5 Log | 1 | minute |
| E. hirae | 99.999% | 5 Log | 1 | minute |
| P. aeruginosa | 99.999% | 5 Log | 1 | minute |
| A. baumannii | 99.999% | 5 Log | 1 | minute |
| Mycobacteria | | | | |
| M. avium | 99.99% | 4 Log | 5 | minutes |
| M. terrae | 99.99% | 4 Log | 5 | minutes |
| Fungi | | | | |
| A. brassiliensis | 99.99% | 4 Log | 5 | minutes |
| C. albicans | 99.99% | 4 Log | 5 | minutes |
| Viruses | | | | |
| Poliovirus type 1 | 99.99% | 4 Log | 15 | minutes |
| Murine norovirus | 99.99% | 4 Log | 15 | minutes |
| Adenovirus type 5 | 99.99% | 4 Log | 15 | minutes |
| Coronavirus Sars - CoV - 2 | 99.99% | 4 Log | 15 | minutes |

The composition of the invention comprises herbal synthesized silver nanoparticie in the range from 5 to 25 nm, and its BET report output is given as a chart.

### Chart 1: Nanoparticles Surface Area Analysis Report (BET)

Summary Report

Surface Area
Single point surface area at P/Po=0.299141472:156.2584 m²/g
BET Surface Area: 154.4047 m²/g
Langmuir Surface Area: 357.7420 m²/g
t-Plot External Surface Area: 201.5669 m²/g
BJH Adsorption cumulative surface area of pores between 17.000 A and 3000.000 A width: 128.913 m²/g Pore Volume
t-Plot micropore volume: −0.019883 cm³/g
BJH Adsorption cumulative volume of pores between 17.000 A and 3000.000 A width: 0.079199 cm³/g Pore Size
BJH Adsorption average pore width (4V/A): 24.574 A

In this manner, the product can be used as broad effect disinfectant substance for different purposes in different fields. Herbal synthesis disinfectant composition of the invention for providing personal hygiene with general and public areas, especially the health sector being in the first place will be used:
In dentistry and dental hospitals; in the disinfection of implant and prosthesis surfaces,
In social areas; in the disinfection of schools, nurseries, kindergartens, sports halls, hotels, job sites and mosques;
In the disinfection of mass transportation and privately owned vehicles;
In homes; in providing hygiene of living areas and personal hygiene,
In the disinfection of air conditioning and ventilation systems;
In drug industry; against bacteria resistant to existing antibiotics,
In food sector; in providing hygiene of packaging and food hygiene,
In the disinfection of public area floor surfaces;
In the disinfection of hospital floor surfaces;
In the disinfection of intensive care room, surgery room, delivery room;
In the disinfection of medical equipment;
In the disinfection of public areas such as schools, mosques, municipality buildings, hospitals, subways, buses;
playgrounds; In the disinfection of areas wherein children are present such as kindergartens, playgrounds, nurseries;
In the disinfection of military, personal care and public areas;
animal farms; to provide disinfection of animals and the workers,
vehicle cleaning; in the cleaning of glasses, in removal of all kinds of dust or dirt on the wheel rim and vehicle surface, in the cleaning of seats in the car and places like ceiling, etc.,
food packaging and storage; in providing hygiene conditions in food packagings or storage areas;
personal care; in care products such as wet wipes, diapers, soaps, hand disinfectants, tooth pastes, creams, etc.; —it prevents formation of bacteria which tend to produce in diapers or wet wipes. It prevents decay on the teeth especially when used inside tooth paste and treats the wounds inside the mouth—
cosmetics; in the field of cosmetics as an antimicrobial for products such as lipsticks, blushers, eye shadows in which bacteria grow in time depending on the usage;
in pool hygiene; —it is mentioned by the doctors that chlorous disinfectants trigger some health problems, and increase middle ear ailment especially in children. And in pool disinfection having biological nanoparticles such health problems are not encountered. Also, infectious diseases such as mycosis which are possible to develop in pools do not show up—
sterile areas; in the sterilization of places such as hospitals and drug production areas;
airlines; in the disinfection of airplanes, passenger waiting halls, ground services offices;
In obtaining antimicrobial products in textile and glass industries.

In usage, for floor surface disinfection, sufficient amount of composition is sprayed, distributed to the surface with the aid of a cloth by rubbing, it does not require rinsing. And for environment disinfection, sufficient amount of product is applied by spraying or with the aid of ULV device. The environment can be used a few minutes after the application, it does not require ventilation. For tool and equipment disinfection, the materials are left inside the product which is put into the immersion container, taken out and dried a few minutes later or the product is applied over the tools and equipment in the form of direct spraying and left to dry. It does not require rinsing. It is used by adding to the finish liquor or by directly spraying and curing to the glass surface in obtaining antimicrobial effect textile and glass products. The composition of the invention can be applied as needed depending on the microbic load of the environment.

1. A nano-encapsulated disinfectant composition comprising herbal synthesized silver nanoparticles, the composition further comprising myrrh tree plant extract, highbush cranberry leaf extract, sumac leaf extract, juniper fruit extract, jasmine and/or daisy flower extract, apple and sunflower face pomace, silver nanoparticle and pure water.

2. The composition according to claim 1, comprising 1-2.5% by weight myrrh tree plant extract, 0.7-1.3% by weight highbush cranberry leaf extract, 0.6-2.2% by weight sumac leaf extract, 0.3-0.9% by weight jasmine and/or daisy flower extract, 2-4.5% by weight juniper fruit extract, 0.74-1.2% by weight apple pomace, 0.9-1.4% by weight sunflower face pomace, 0.037-0.059% by weight silver nanoparticle and 86-93% by weight pure water.

3. The composition according to claim 1, comprising 2.067% by weight myrrh tree plant extract, 1.1% by weight highbush cranberry leaf extract, 1.5% by weight sumac leaf extract, 1% by weight jasmine or daisy flower extract, 3.4% by weight juniper fruit extract, 0.98% by weight apple pomace, 1.25% by weight sunflower face pomace, 0.044% by weight silver nanoparticle and 88.659% by weight pure water.

4. A production method of a nano-encapsulated disinfectant composition comprising herbal synthesized silver nanoparticles wherein the method comprises the following process steps:
i. Brewing myrrh tree, highbush cranberry leaf and sumac leaf in water,
ii. Extraction of juniper fruits in water,
iii. Extraction of jasmine and/or daisy plants in water/vegetative alcohol,
iv. Gelling apple and sunflower face pomace in water, obtaining pectin with lactic and citric acid,
v. Preparation of solution with pure silver, acetic acid and hydrogen peroxide,
vi. Mixing the prepared solution with the vegetative extracts obtained in process steps (i), (ii), (iii), and
vii. Coating the nanoparticle obtained in step (vi) with the nano-capsule material obtained in step (iv).

5. The method according to claim 4, wherein process step (i) comprises the processes of brewing the Myrrh tree plant in pure water first at 65-75° C. for 20-25 minutes by occasionally mixing; brewing at 65-85° C. for 10-15 minutes by occasionally mixing by adding equal amounts of powdered Highbush cranberry leaf and Sumac plants over it; subjecting to ultrasonification process in an ultrasonic device at 40° C. for 10-15 minutes for increasing the speed of extraction; shock cooling of the obtained extract and subjecting to ultrasonification process for 10-15 minutes; subjecting to shock cooling process and centrifuging at +1° C. at 4500-5500 rpm for 10-15 minutes and storing at +4° C.

6. The method according to claim 4, wherein process step (ii) comprises the processes of heating Juniper fruits in pure water first at 85-95° C. for 20-30 minutes; subjecting to ultrasonification process for 10-15 minutes, then leaving it to fermentation in the closed container at 45-55° C. for 5-10 days; shock cooling of the obtained extract; and centrifuging at +1° C. at 4500-5500 rpm for 10-15 minutes and storing at +4° C.

7. The method according to claim 4 wherein process step (iii) comprises the processes of brewing jasmine (and/or daisy) flowers first at 35-45° C. for 5-10 minutes by adding 54% vegetative alcohol; then at 50-60° C. for 20-30 minutes by adding pure water by occasionally mixing; cooling in ice bath by subjecting to shock cooling and subjecting the cooled extract to ultrasonification process for 10-15 minutes in ultrasonic device; centrifuging at +1° C. at 4500-5500 rpm for 5-7 minutes; separation of solid and liquid extract parts by filtering through filter paper having 11 μm pore diameter.

8. The method according to claim 4 wherein process step (v) comprises the processes of weighing pure silver in the ratio of 0.037-0.059% and treating with acetic acid and hydrogen peroxide in the ratio of 0.5:2; adding the extracts obtained in process steps (i) (ii) to the solution in bottle number 5 and mixing at 750-850 rpm for 5-10 minutes, and addition of the extract obtained in process step (iii) and mixing at 750-850 rpm for 20-30 minutes.

9. The method according to claim 4 wherein process step (iv) comprises the processes of putting 200-350 grams of apple pomace and adding 20-30 ml of pure water over it; smashing for 1-3 minutes, mixing at 250-300 rpm to bring to boiling point at 80-90° C. for 25-30 minutes; adding citric and lactic acid over it and mixing and cooling; centrifuging at 5300 rpm for 10-15 minutes and filtering through filter paper and leaving at 60-80° C. for 20-25 minutes.

10. The method according to claim 4 wherein process step (iv) comprises the processes of putting 150-250 grams of sunflower face pomace and adding 400-500 ml of pure water over it; heating at 90-95° C. for 35-40 minutes; addition of citric and lactic acid and mixing continuously at 250-350 rpm and smashing for 1-3 minutes; cooling for 20-30 minutes and centrifuging at 5300 rpm for 10-15 minutes; filtering through filter paper and heating at 30-40° C. for 20-25 minutes.

11. The method according to claim 4 wherein process step (vii) comprises the processes of taking the supernatant obtained in process step (iv) from apple pomace and adding dropwise slowly to the mixture obtained in process step (v) which is mixed continuously at 40° C.

12. The method according to claim 4 wherein process step (vii) comprises the processes of taking the supernatant obtained in process step (iv) from sunflower face pomace and adding dropwise slowly to the mixture obtained in process step (v) which is mixed continuously at 40° C.
